(19) United States
(12) Patent Application Publication
(10) Pub. No.: US 2025/0243441 A1
(43) Pub. Date: Jul. 31, 2025

(54) BIONIC ORGAN DEVICE

(71) Applicant: DARWIN PRECISIONS CORPORATION, Hsinchu County (TW)

(72) Inventors: Ting-Chieh Yang, Hsinchu County (TW); Chung-Yi Huang, Hsinchu County (TW)

(21) Appl. No.: 19/021,270

(22) Filed: Jan. 15, 2025

(30) Foreign Application Priority Data
Jan. 30, 2024 (TW) 113103554

(51) Int. Cl. C12M 1/12; C12M 1/42

(52) U.S. Cl. CPC C12M 25/04; C12M 35/02

(57) ABSTRACT

A bionic organ device includes an organ chip and a power module. The organ chip includes a first body, a second body, a porous film and a piezoelectric element. The porous film is disposed between the first body and the second body and forms a flow channel system with the first body and the second body. The flow channel system includes a first passage and a second passage. The first passage is located between the first body and the porous film, and the second passage is located between the second body and the porous film. The piezoelectric element is disposed on one side of the porous film and is connected to the porous film and is electrically connected to the power module. The power module drives the piezoelectric element to deform, and the deformation of the at least one piezoelectric element drives the size of the porous film to change.

### FIELD OF THE INVENTION

The present invention relates to a bionic technology, and in particular to a bionic organ device capable of simulating a micro-environment within an organism.

### BACKGROUND OF THE INVENTION

Conventional cell culture models cannot reflect the complex physiological functions of tissues and organs of organisms, while animal experiments have disadvantages such as long cycles and high costs. Moreover, it is always difficult to directly predict the real reactions of organisms. The organ chip mimics key functions of an organism organ, reconstructs a physiological environment of an organ in vivo, simulates a structure, a micro-environment, and physiological functions of the organism organ, and accurately controls parameters. In addition, the organ chip has advantages such as miniaturization, integration, high efficiency, and reduced costs. Furthermore, to simulate the stretching and shrinking of organ cells, the existing organ chip is provided with a vacuum system, which performs vacuum suction to stretch cells, thereby achieving a bionic effect. However, vacuum stretching also pulls a membrane to which cells attach, causing membrane damage and an organ chip malfunction. The manufacturing process of the vacuum system is complex and therefore requires improvement.

### SUMMARY OF THE INVENTION

The present invention provides a bionic organ device, which can be used to simulate a dynamic micro-environment of an organ and has a simplified structure conducive to simplifying the manufacturing process, reducing the costs, and improving the yield.

The bionic organ device provided by the present invention includes an organ chip and a power module. The organ chip includes a first body, a second body, a porous film, and at least one piezoelectric element. The porous film is disposed between the first body and the second body and forms a flow channel system with the first body and the second body. The flow channel system includes a first passage and a second passage. The first passage is located between the first body and the porous film, and the second passage is located between the second body and the porous film. The at least one piezoelectric element is disposed on at least one side of the porous film and is connected to the porous film, and is electrically connected to the power module. The power module is used to drive the at least one piezoelectric element to deform, and the deformation of the at least one piezoelectric element drives the size of the porous film to change.

With the use of the piezoelectric element and the porous film, the organ chip of the present invention can be used to simulate the stretching or shrinking of organs, tissues, or cells, and is convenient during use. Moreover, the organ chip of the present invention has a simplified structure and thus is conducive to simplifying the manufacturing process, reducing the costs, and improving the yield.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic three-dimensional diagram of a bionic organ device according to an embodiment of the present invention;

FIG. 2 is a partially schematic three-dimensional exploded view of a bionic organ device according to an embodiment of the present invention;

FIG. 3 is a schematic cross-sectional view of the bionic organ device, taken along the line A-A in FIG. 1;

FIG. 4 is a partially schematic three-dimensional exploded view of a bionic organ device according to another embodiment of the present invention;

FIG. 5 is a schematic top view of a piezoelectric element according to an embodiment of the present invention;

FIG. 6 is a schematic top view of a piezoelectric element and a deformed porous film according to an embodiment of the present invention; and

FIG. 7 is another schematic top view of the piezoelectric element and the deformed porous film according to an embodiment of the present invention.

### DETAILED DESCRIPTION OF PREFERRED EMBODIMENTS

The foregoing and other technical contents and other features and advantages of the present invention will be clearly presented from the following detailed description of a preferred embodiment in cooperation with the accompanying drawings. Directional terms mentioned in the following examples, for example, upper, lower, left, right, front, back, top, or bottom, are only used to describe directions referring to the attached drawings. Therefore, the directional terms used are for illustration and not for limitation. In addition, terms such as “first” and “second” involved in the description or claims are merely used for naming the elements or distinguishing different embodiments or ranges rather than limiting the upper limit or lower limit of the quantity of the elements.

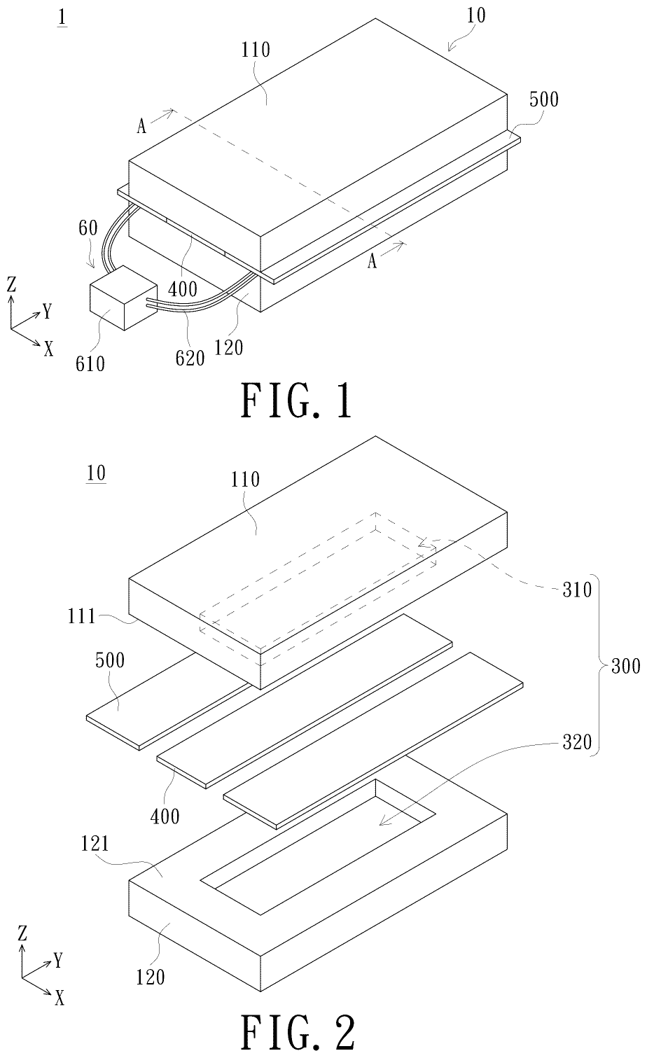

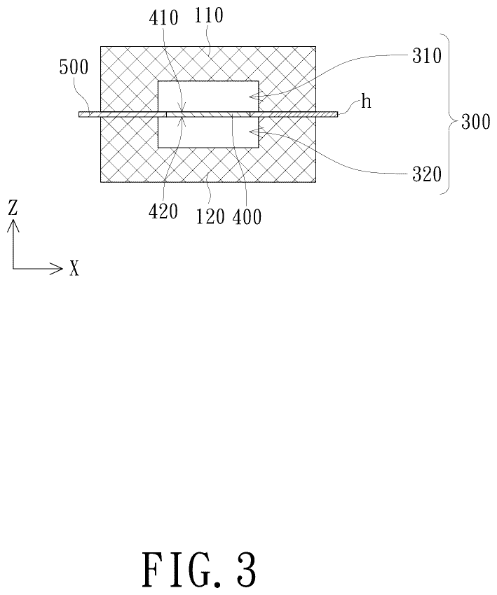

FIG. 1 is a schematic three-dimensional diagram of a bionic organ device according to an embodiment of the present invention. FIG. 2 is an exploded view of the bionic organ device shown in FIG. 1. FIG. 3 is a schematic cross-sectional view of the bionic organ device, taken along the line A-A in FIG. 1. As shown in FIGS. 1 to 3, the bionic organ device of the embodiments of the present invention includes an organ chip 10 and a power module 60, and the power module 60 is electrically connected to the organ chip 10. The organ chip 10 includes a first body 110, a second body 120, a porous film 400, and at least one piezoelectric element 500. The porous film 400 is disposed between the first body 110 and the second body 120, and the at least one piezoelectric element 500 is connected to the porous film 400 and electrically connected to the power module 60.

As shown in FIGS. 2 and 3, the first body 110 and the second body 120 may have a groove-shaped structure, which has an accommodating space and an opening. The opening of the first body 110 is opposite the opening of the second body 120. The first body 110, the second body 120, and the porous film 400 form the main parts of the organ chip 10. At least one side of the porous film 400 is connected to the piezoelectric element 500. The porous film 400 may be connected to, via the piezoelectric element 500, the surface 111 of the first body 110 facing the second body 120 and the surface 121 of the second body 120 facing the first body 110.

The porous film 400 forms a flow channel system 300 with the first body 110 and the second body 120. The flow channel system 300 includes a first passage 310 located between the first body 110 and the porous film 400 and a second passage 320 located between the second body 120 and the porous film 400. The flow channel system 300 may further include an input/injection pore (not shown in the figure), which can communicate with the first passage 310 and/or the second passage 320. The input/injection pore can be provided at the organ chip 10 by any known means. For example, it can be formed at the first body 110 and/or the second body 120, further enabling communication between the inside and the outside of the organ chip 10.

The first passage 310 and/or the second passage 320 may be used for at least one fluid (not shown in the figure) to pass through or stay therein, and two opposite sides of the porous film 400 have film surfaces. The first film surface 410 is located at the first passage 310, the second film surface 420 is located at the second passage 320, and the fluid may be in contact with the first film surface 410 and the second film surface 420 and may further cover them. The porous film 400 may have, for example, a three-dimensional support structure or a mesh-like structure, which is flexible, stretchable, and extendable. The pore size of the porous film 400 may be in units of nanometer (nm) and cover a range of, for example, tens of nanometers, dozens of nanometers, or hundreds of nanometers. The porous film 400 may be made of, for example, polyethylene terephthalate (PETE), polydimethylsiloxane (PDMS), polyurethane, styrene-ethylene-butylene-styrene (SEBS), poly(hydroxyethyl methacrylate) (pHEMA), polyethylene glycol or polyethylene glycol, polycarbonate (PC), or may be made of another natural material or an artificial synthetic polymer material. In several embodiments of the present invention, the porous film 400 may be a hydrophilic polymer material including, for example, but not limited to a polysaccharide: cellulose, starch, hyaluronic acid, alginate, or chitosan; a polypeptide: collagen, poly-L-lysine, or poly-L-glutamic acid; or an artificial synthetic polymer: polyacrylic acid, polymethacrylic acid, or polyacrylamide.

The porous film 400 may serve as a cell attachment membrane for cells to attach to the first film surface 410 and/or the second film surface 420 to further culture the cells in the foregoing fluid. The same or different cells can attach to the first film surface 410 and the second film surface 420, and the fluids in the first passage 310 and the second passage 320 can vary based on the types of the cells. For example, in several embodiments of the present invention, the first film surface 410 is used for the attachment of alveolar epithelial cells, and the second film surface 420 is used for the attachment of microvascular endothelial cells. Accordingly, the first passage 310 is supplied with an oxygen-containing gas, while the second passage 320 is supplied with a culture solution.

As described above, at least one side of the porous film 400 is connected to the piezoelectric element 500. The piezoelectric element 500 is an element that can deform through the application of voltage or an element that can generate a voltage after a force (pressure) is applied to it. In a preferred embodiment of the present invention, the piezoelectric element 500 can deform through the application of voltage, and the power module 60 electrically connected therewith can drive the piezoelectric element 500 to deform. In addition, the connection between the piezoelectric element 500 and the porous film 400 can be achieved using one or more known methods such as hot pressing, melting, bonding, or another achievable method. In several embodiments of the present invention, the piezoelectric element 500 and the porous film 400 are connected using a thermosetting or thermoplastic material, such as a hydrogel. When the piezoelectric element 500 deforms, it drives the porous film 400 connected therewith so as to pull or push the porous film 400, thus changing the size of the porous film 400.

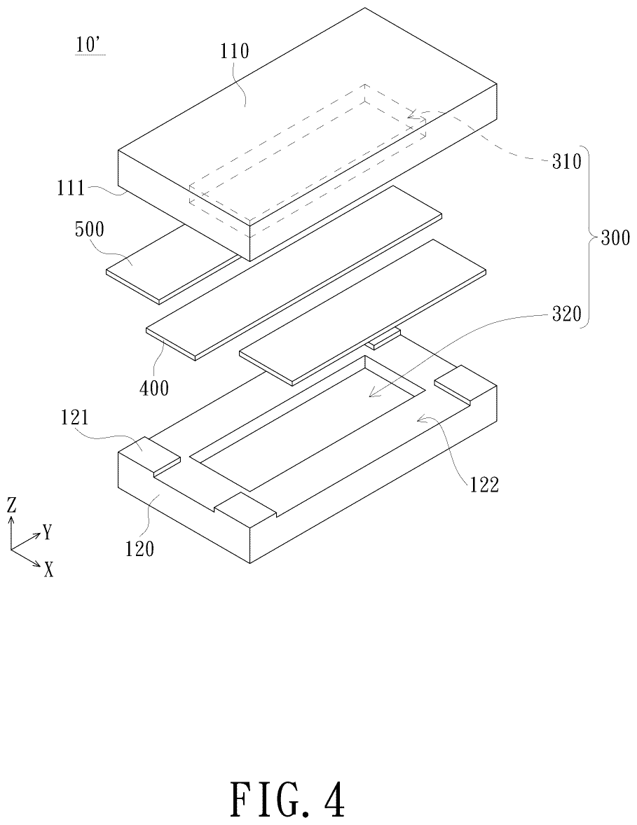

As shown in FIGS. 2 and 3, the piezoelectric element 500 may be disposed between the first body 110 and the second body 120. In addition, the piezoelectric element 500 can abut against the surface 111 of the first body 110 and the surface 121 of the second body 120. The piezoelectric element 500 may also be connected to the first body 110 or the second body 120 through one or more known methods, such as bonding, locking, or another method. In several embodiments of the present invention, the organ chip 10 may be further provided with an accommodating space for arranging the piezoelectric element 500. The accommodating space may be, for example, a groove or a channel and formed on a surface 111 of the first body 110 and/or a surface 121 of the second body 120. FIG. 4 is a schematic exploded view of an organ chip of a bionic organ device according to another embodiment of the present invention. As shown in FIG. 4, the organ chip 10′ has an accommodating space 122 formed on the surface 121 of the second body 120, and the piezoelectric element 500 is disposed in the accommodating space 122. In addition, in other embodiments of the present invention, the first body 110 may be also provided with an accommodating space. The accommodating spaces of the first body 110 and the second body 120 may be separate or combined for accommodating the piezoelectric element 500.

In the embodiments of the present invention, the deformation of the piezoelectric element 500 may include a change in its size, and the change in size may further include a change in the length of the piezoelectric element 500 in any direction. The deformation amount may be, for example, 0%-10%. As described above, the piezoelectric element 500 can abut against the surface 111 of the first body 110 and the surface 121 of the second body 120; and, preferably, the deformation does not abut against the first body 110 and the second body 120 on two opposite sides when the piezoelectric element 500 deforms. In other words, in the embodiments of the present invention, the deformation of the piezoelectric element 500 such as an increase or decrease in volume or an increase or decrease in length occurs in other directions than the first direction Z in which the first body 110 is connected to the second body 120. In addition, the deformation direction of the piezoelectric element 500 is not limited. For example, the piezoelectric element 500 can deform in the second direction X, the third direction Y, and a combination thereof. The second direction X and the third direction Y may be perpendicular to the first direction Z and substantially parallel to the surface of the porous film 400. When the piezoelectric element 500 deforms in the second direction X, the porous film 400 can be preferably driven in the second direction X, such that the porous film 400 can stretch or shrink. When the piezoelectric element 500 deforms in the third direction Y, the porous film 400 can be driven in the third direction Y. In the embodiments of the present invention, the stretching or shrinking of the porous film 400 allows for a change amount of 0%-10% in the length compared with the original length.

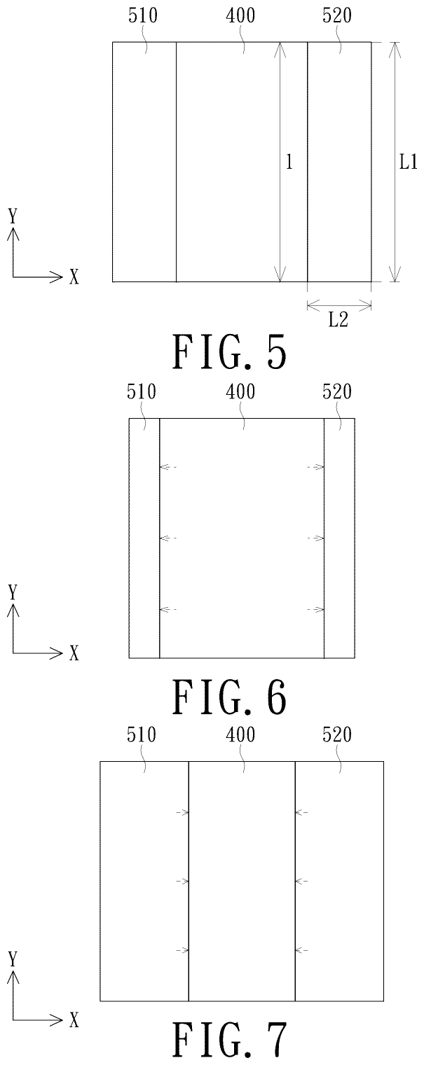

FIG. 5 is a schematic top view of a piezoelectric element according to an embodiment of the present invention. As shown in FIGS. 2, 3, and 5, two piezoelectric elements 500 are provided, including a first piezoelectric element 510 and a second piezoelectric element 520. The first piezoelectric element 510 and the second piezoelectric element 520 each may be roughly in the shape of a rectangular sheet or a rectangular plate and have a first edge length L1, a second edge length L2, and a thickness h. The direction of the thickness h is substantially parallel to the first direction Z and the direction of the first edge length L1 is perpendicular to the second direction X. In this embodiment, the side of the piezoelectric element 500 having the first edge length L1 is connected to the porous film 400. The first piezoelectric element 510 and the second piezoelectric element 520 are respectively located on two opposite sides of the porous film 400, and the side of the porous film 400 connected to the first piezoelectric element 510 or the second piezoelectric element 520 has a length I. Preferably, the first edge length L1 is substantially the same as the length I, but this is not limited thereto. In this embodiment, when the piezoelectric element 500 deforms in the second direction X, a side of the porous film 400 connected to the piezoelectric element 500 is pulled or pushed, such that the porous film 400 stretches or shrinks.

FIGS. 6 and 7 are schematic top views of a piezoelectric element and a deformed porous film according to an embodiment of the present invention. As shown in FIG. 6, in several embodiments, the piezoelectric element 500 shrinks in volume and decreases in length in the first direction X under a voltage such as a first voltage value, thus pulling the porous film 400 to stretch (an arrow indicates the stretching action of the porous film 400). In addition, as shown in FIG. 7, in several embodiments, the piezoelectric element 500 becomes large in volume and increases in length in the first direction X under another voltage such as a second voltage value, thus pushing the porous film 400 to shrink (an arrow indicates the shrinking action of the porous film 400). The first voltage value and the second voltage value can be provided by a power module 60, and in a preferred embodiment of the present invention, the two are opposite and different. For example, one is a positive value, and the other is a negative value. When the first voltage value or the second voltage value is applied, at intervals, to the piezoelectric element 500, the porous film 400 can stretch or shrink periodically and therefore serves as a cell attachment membrane allowing cells to stretch or shrink in an internal environment of an organism. The first voltage value and the second voltage value may change alternately, allowing the porous film 400 to periodically stretch and shrink.

The material of the piezoelectric element 500 includes a natural or synthetic inorganic compound, a metal oxide, a metal nitride, an acidic oxide, a polymer compound, or a combination thereof. The material is, for example, but not limited to a piezoelectric single crystal such as quartz or lithium niobate (LiNbO₃); a piezoelectric ceramic such as barium titanate (BaTiO₃), sodium bismuth titanate ((Bi,Na)TiO₃), lead zirconate titanate (PZT), or a lead, zirconium, titanium, barium oxide; a piezoelectric thin film such as zinc oxide (ZnO), aluminum nitride (AlN), or lead zirconate titanate (PZT); and a piezoelectric polymer film such as [P(VDF-TrFE)]. The shape of the piezoelectric element 500 is not limited to the foregoing rectangular sheet and the rectangular plate, and for example, may be in various shapes due to different materials. In addition, a proper shape can be formed using a powdered piezoelectric material. In several embodiments of the present invention, the shape of the piezoelectric element 500 may be, for example, a rod or a pillar, to fit with the first body 110 and the second body 120.

In the embodiments of the present invention, the piezoelectric element 500 may have a deformation amount of, for example, 0%-10%, and the deformation amounts of the porous film 400 and the piezoelectric element 500 may be the same or different. For example, when the deformation amounts of the porous film 400 and the piezoelectric element 500 are substantially the same and the piezoelectric elements 500 on two opposite sides of the porous film 400 such as the first piezoelectric element 510 and the second piezoelectric element 520 each increase by 10 μm in the second direction X, the porous film 400 can be also stretched by 10 μm in the second direction X, but this is not limited thereto. In another embodiment, the piezoelectric element 500 may be disposed on only one side of the porous film 400. When its length increases by 10 um, the porous film 400 can be stretched by 5 μm, but this is not limited thereto. In several embodiments of the present invention, the deformation amount of the piezoelectric element 500 may be also determined based on the requirement for the deformation amount of the porous film 400, for example, a change amount of 0%-10% in length of the porous film 400.

As described above, the power module 60 is electrically connected to the piezoelectric element 500, the piezoelectric element 500 may deform through the application of voltage, and the power module 60 can drive the piezoelectric element 500 to deform. The power module 60 may be connected to the piezoelectric element 500 via, for example, a wire material. In a preferred embodiment of the present invention, the power module 60 includes a power supply 610 and at least one wire set 620. The at least one wire set 620 is connected to the piezoelectric element 500, and the power supply 610 is located outside the organ chip 10 and configured to apply a voltage to the piezoelectric element 500, allowing it to deform due to the voltage. As shown in FIG. 1, the wire set 620 can extend from one end of the piezoelectric element 500 to be connected to the power supply 610, but this is not limited thereto. The power supply 610 may output at least one voltage value including, for example, a first voltage value, a second voltage value, or both, and the first voltage value is different from the second voltage value. When the power supply 610 does not apply a voltage to the piezoelectric element 500, the outputted voltage value may be considered as 0 or no voltage is outputted.

For example, the power supply 610 may output a first voltage value, a second voltage value, or a third voltage value, the three voltage values are different, but one of them is zero. For ease of description, the third voltage value is zero by default. Under the third voltage value, the piezoelectric element 500 has no deformation amount, and the porous film 400 does not stretch or shrink. Under the first voltage value, for example, the piezoelectric element 500 shrinks in volume and the porous film 400 stretches. On the contrary, under the second voltage value, the piezoelectric element 500 becomes large in volume and the porous film 400 shrinks. The first voltage value, the second voltage value, and the third voltage value may be in units of volt (V), with a specific value varying depending on a piezoelectric material. For example, in several embodiments of the present invention, under the combination of the piezoelectric ceramic, the outputted voltage value may be, for example, −110 V to 110 V.

The power supply 610 may alternately output any two of the first voltage value, the second voltage value, and the third voltage value. For example, the first voltage value and the second voltage value are alternately outputted to enable the porous film 400 to stretch and shrink alternately, or the first voltage value and the third voltage value are alternately outputted to enable the porous film 400 to stretch periodically. Therefore, when the porous film 400 serves as a cell attachment membrane, the stretching or shrinking of cells in the environment of an organism is enabled. The combination use with the flow channel system 300 allows for a more realistic simulation of the dynamic micro-environment of an organ.

With the use of the piezoelectric element 500, the present invention provides a method, for simulating stretching/shrinking of organs, tissues, or cells, completely different from the conventional vacuum method. Stretching/shrinking of organs, tissues, or cells can be simulated by setting the power supply 610 outside the organ chip 10 by a user. This, compared with extracting or delivering gas by a vacuum system, is more convenient. In addition, the conventional vacuum system needs to be provided with a passage for extracting and delivering gas in the organ chip, so as to complete simulation. Under comparison, the organ chip 10 of the present invention does not need a vacuum system and therefore has a simplified structure and manufacturing process, which is conducive to reducing costs and improving the yield.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A bionic organ device, comprising an organ chip and a power module, wherein the organ chip comprises a first body, a second body, a porous film, and at least one piezoelectric element, wherein
the porous film is disposed between the first body and the second body and forms a flow channel system with the first body and the second body, and the flow channel system comprises:
a first passage, located between the first body and the porous film; and
a second passage located between the second body and the porous film;
wherein the at least one piezoelectric element is disposed on at least one side of the porous film, connected to the porous film, and electrically connected to the power module,
wherein the power module is configured to drive the at least one piezoelectric element to deform, and the deformation of the at least one piezoelectric element drives a size of the porous film to change.

2. The bionic organ device according to claim 1, wherein the power module is configured to apply a voltage to the at least one piezoelectric element, and the at least one piezoelectric element deforms by the voltage.

3. The bionic organ device according to claim 1, wherein the porous film is used as a cell attachment membrane, the porous film has a first film surface and a second film surface, the first film surface is located at the first passage, the second film surface is located at the second passage, and the first film surface, the second film surface, or a combination of the first film surface and the second film surface is used for cells to attach.

4. The bionic organ device according to claim 1, wherein the first passage, the second passage, or a combination of the first passage and the second passage is used for at least one fluid to pass therethrough, and the at least one fluid is in contact with at least one film surface of the porous film.

5. The bionic organ device according to claim 1, wherein the at least one piezoelectric element is further disposed between the first body and the second body, the first body is connected to the second body in a first direction, and a deformation of the at least one piezoelectric element occurs in a direction different from the first direction.

6. The bionic organ device according to claim 5, wherein the deformation of the at least one piezoelectric element occurs in a second direction, the second direction is perpendicular to the first direction, and the deformation of the at least one piezoelectric element drives the porous film to stretch or shrink in the second direction.

7. The bionic organ device according to claim 5, wherein the deformation of the at least one piezoelectric element comprises a change in size of the at least one piezoelectric element.

8. The bionic organ device according to claim 5, wherein the deformation of the at least one piezoelectric element comprises a change in length of the at least one piezoelectric element in the direction.

9. The bionic organ device according to claim 6, wherein the porous film has a length on the at least one side, the at least one piezoelectric element is rectangular, sheet-like, plate-like, or columnar and has a thickness and an edge length, a direction of the edge length is different from the second direction, a side of the at least one piezoelectric element having the edge length is connected to the porous film, and the edge length is substantially the same as the length of the porous film.

10. The bionic organ device according to claim 1, wherein the at least one piezoelectric element comprises a first piezoelectric element and a second piezoelectric element, respectively disposed on two opposite sides of the porous film.

11. The bionic organ device according to claim 2, wherein the power module comprises a power supply and at least one wire set, the at least one wire set is connected to the at least one piezoelectric element, and the power supply applies a voltage to the at least one piezoelectric element via the at least one wire set.

12. The bionic organ device according to claim 11, wherein the power supply outputs at least one voltage value, the at least one voltage value comprises a first voltage value, a second voltage value, or a combination of the first voltage value and the second voltage value, the first voltage value is different from the second voltage value, and at least one of the first voltage value and the second voltage value is not zero.

13. The bionic organ device according to claim 1, wherein the first body has a lower surface, the second body has an upper surface, the first body is connected to the second body with the lower surface facing the upper surface, the organ chip further comprises an accommodating space formed by the upper surface, the lower surface, or a combination of the upper surface and the lower surface, and the at least one piezoelectric element is disposed in the accommodating space.

14. The bionic organ device according to claim 13, wherein the accommodating space comprises a groove, a channel, or a combination of the groove and the channel.

15. The bionic organ device according to claim 1, wherein a material of the piezoelectric element comprises a natural or synthetic inorganic compound, a metal oxide, a metal nitride, an acidic oxide, a polymer compound, or a combination thereof.
